(19) United States
(12) Patent Application Publication
(10) Pub. No.: US 2014/0263220 A1
(43) Pub. Date: Sep. 18, 2014

(54) LASER CUTTING PROCESS MONITORING AND CONTROL

(71) Applicant: ABBOTT CARDIOVASCULAR SYSTEMS INC., Santa Clara, CA (US)

(72) Inventors: Li Chen, San Jose, CA (US); Yongjin Xie, Cupertino, CA (US)

(73) Assignee: ABBOTT CARDIOVASCULAR SYSTEMS INC., Santa Clara, CA (US)

(21) Appl. No.: 14/212,086

(22) Filed: Mar. 14, 2014

Related U.S. Application Data
(60) Provisional application No. 61/798,490, filed on Mar. 15, 2013.
(60) Provisional application No. 61/801,565, filed on Mar. 15, 2013.

(51) Int. Cl. B23K 26/03; A61F 2/91

(52) U.S. Cl. CPC B23K 26/032; A61F 2/91
USPC 219/121.72; 219/121.67

(57) ABSTRACT

A laser system including various optical components providing for beam alignment and process monitoring of a stent cutting process is described. The various aspects of the invention provide for monitoring of a beam of laser light reflected from the surface of a stent tube so as monitor the properties of the cutting beam, location of the beam, system cleanliness, optical defects in the system, and cutting efficiency of the laser.

### CROSS-REFERENCES TO RELATED APPLICATIONS

This application claims priority from U.S. Application No. 61/798,490, filed Mar. 15, 2013 and 61/801,565, filed Mar. 15, 2013 incorporated by reference in its entirety.

### BACKGROUND

1. Field of the Invention

The present invention relates generally to implantable medical devices and to a method for manufacturing implantable medical devices. These implantable medical devices may also be capable of retaining therapeutic materials and dispensing the therapeutic materials to a desired location of a patient's body. More particularly, the present invention relates to a system and method for forming the structure of a stent or intravascular or intraductal medical device, and for monitoring and controlling the process used to form the stent structure.

2. General Background and State of the Art

In a typical percutaneous transluminal coronary angioplasty (PTCA) for compressing lesion plaque against the artery wall to dilate the artery lumen, a guiding catheter is percutaneously introduced into the cardiovascular system of a patient through the brachial or femoral arteries and advanced through the vasculature until the distal end is in the ostium. A dilatation catheter having a balloon on the distal end is introduced through the catheter. The catheter is first advanced into the patient's coronary vasculature until the dilatation balloon is properly positioned across the lesion.

Once in position across the lesion, a flexible, expandable, preformed balloon is inflated to a predetermined size at relatively high pressures to radially compress the atherosclerotic plaque of the lesion against the inside of the artery wall and thereby dilate the lumen of the artery. The balloon is then deflated to a small profile, so that the dilatation catheter can be withdrawn from the patient's vasculature and blood flow resumed through the dilated artery. While this procedure is typical, it is not the only method used in angioplasty.

In angioplasty procedures of the kind referenced above, restenosis of the artery often develops which may require another angioplasty procedure, a surgical bypass operation, or some method of repairing or strengthening the area. To reduce the likelihood of the development of restenosis and strengthen the area, a physician can implant an intravascular prosthesis, typically called a stent, for maintaining vascular patency. In general, stents are small, cylindrical devices whose structure serves to create or maintain an unobstructed opening within a lumen. The stents are typically made of, for example, stainless steel, nitinol, or other materials and are delivered to the target site via a balloon catheter. Although the stents are effective in opening the stenotic lumen, the foreign material and structure of the stents themselves may exacerbate the occurrence of restenosis or thrombosis.

A variety of devices are known in the art for use as stents, including expandable tubular members, in a variety of patterns, that are able to be crimped onto a balloon catheter, and expanded after being positioned intraluminally on the balloon catheter, and that retain their expanded form. Typically, the stent is loaded and crimped onto the balloon portion of the catheter, and advanced to a location inside the artery at the lesion. The stent is then expanded to a larger diameter, by the balloon portion of the catheter, to implant the stent in the artery at the lesion. Typical stents and stent delivery systems are more fully disclosed in U.S. Pat. No. 5,514,154 (Lau et al.), U.S. Pat. No. 5,507,768 (Lau et al.), and U.S. Pat. No. 5,569,295 (Lam et al.).

Stents are commonly designed for long-term implantation within the body lumen. Some stents are designed for non-permanent implantation within the body lumen. By way of example, several stent devices and methods can be found in commonly assigned and common owned U.S. Pat. No. 5,002,560 (Machold et al.), U.S. Pat. No. 5,180,368 (Garrison), and U.S. Pat. No. 5,263,963 (Garrison et al.).

Intravascular or intraductal implantation of a stent generally involves advancing the stent on a balloon catheter or a similar device to the designated vessel/duct site, properly positioning the stent at the vessel/duct site, and deploying the stent by inflating the balloon which then expands the stent radially against the wall of the vessel/duct. Proper positioning of the stent requires precise placement of the stent at the vessel/duct site to be treated. Visualizing the position and expansion of the stent within a vessel/duct area is usually done using a fluoroscopic or x-ray imaging system.

Although PTCA and related procedures aid in alleviating intraluminal constrictions, such constrictions or blockages reoccur in many cases. The cause of these recurring obstructions, termed restenosis, is due to the body's immune system responding to the trauma of the surgical procedure. As a result, the PTCA procedure may need to be repeated to repair the damaged lumen.

In addition to providing physical support to passageways, stents are also used to carry therapeutic substances for local delivery of the substances to the damaged vasculature. For example, anticoagulants, antiplatelets, and cytostatic agents are substances commonly delivered from stents and are used to prevent thrombosis of the coronary lumen, to inhibit development of restenosis, and to reduce post-angioplasty proliferation of the vascular tissue, respectively. The therapeutic substances are typically either impregnated into the stent or carried in a polymer that coats the stent. The therapeutic substances are released from the stent or polymer once it has been implanted in the vessel.

In the past, stents have been manufactured in a variety of manners, including cutting a pattern into a tube that is then finished to form the stent. The pattern can be cut into the tube using various methods known in the art, including using a laser.

Laser cutting of the stent pattern initially utilized lasers such as the Nd:YAG laser, configured either at its fundamental mode and frequency, or where the frequency of the laser light was doubled, tripled or even quadrupled to give a light beam having a desired characteristic to ensure faster and cleaner cuts.

Recently, lasers other than conventional Nd:YAG lasers have been used, such as diode-pumped solid-state lasers that operate in the short pulse pico-second and femto-second domains. These lasers provide improved cutting accuracy, but cut more slowly than conventional lasers such as the long pulse Nd:YAG laser.

Throughout the process of fabricating a stent implant from raw tubing there is a general desire to minimize the amount of contamination in non-beneficial materials affects that can result from the introduction of high heat and formed substances to the tubing. One process that is particularly susceptible to these affects is the laser cutting process, since it introduces both heat in the form of laser energy and foreign materials in the form of shielding gases and surrounding environmental gases into the stent cutting process. Oxygen is a particular concern because it can lead to material oxidation and embrittlement of the material due to reactions between the oxygen and the tubing material in the presence of the heat generated by the laser-cutting beam.

A further concern is the effect of molten stent material and ablated debris generated during the laser cutting process. As tubing is melted or ablated by the laser beam to form a stent structure, the shielding gas, which is typically an inert gas such as argon, directs molten material away from the raw tubing. At least a portion of this material is ejected in a direction that generally opposes the direction of the laser beam. Under certain conditions, such as when the shielding gas is flowing at a low rate, or when the shielding gas nozzle is wide enough to allow entry of particulates, the laser optics can become maned by the escaping particulates. The particulates may deposit on the lens of the laser equipment and over time, these depositions can obscure the path of the laser beam creating detrimental changes the laser beam characteristics. For example, the beam may lose focus, which can result in a less clean cut or longer cutting times. Another concern is the debris from laser cutting may be accumulated inside the cutting kerf, thereby reducing the cutting efficiency of the laser beam.

Another problem with using a laser to cut a stent pattern into a tube is that to ensure that a laser will cut the tubing used to form stents, there must be appropriate laser power to melt or ablate the tubing material. However, when the laser cuts through the tubing, the laser beam may propagate beyond the tubing wall and may melt or burn the opposing wall of the tubing material. This may cause defects in the stent pattern which is ultimately cut from the opposing wall of the tubing.

What has been needed, and heretofore unavailable, is an efficient and cost-effective monitoring system for monitoring and controlling the cutting process that incorporates various features designed to provide for monitoring the cutting beam and optical components of the optical chain to ensure that the laser system is operating at peak efficiency. The present invention satisfies these, and other needs.

### SUMMARY OF THE INVENTION

In its broadest aspect, the invention provides a laser system that includes various optical components that provide for beam alignment and process monitoring of a stent cutting process. The various aspects of the invention provide for monitoring of a beam of laser light reflected from the surface of a stent tube so as monitor the properties of the cutting beam, location of the beam, system cleanliness and optical defects in the system.

In another aspect, the invention provides a system for monitoring the efficiency of a cutting laser by providing for monitoring the completeness of a laser cut through a wall of a stent tube. The various aspects of the invention include focusing a monitoring laser beam on an area of a stent tube into which a stent pattern has been cut, while the stent tube remains in a cutting fixture, and, based upon a characteristic of light reflected from the cut area of the stent tube, determining a degree of cut-through which is related to the cutting efficiency of the cutting laser.

In still another aspect, the invention includes a system for monitoring the cutting of a stent pattern into a stent tube using a laser beam, comprising: a laser for generating a laser beam; a polarizing beam splitter; a quarter wave plate for imparting circular polarization to the laser beam; a focusing lens for focusing the circularly polarized laser beam onto a stent tube into which a stent pattern is to be cut; and a laser beam analyzer for analyzing a laser beam reflected by the stent tube back along the path of the laser beam and into the polarizing beam splitter, where the reflected beam is deflected into the laser beam analyzer.

In one alternative aspect, the laser beam analyzer is a beam mapper. In another alternative aspect, the laser beam analyzer is a laser cam. In still another alternative aspect, the laser beam analyzer is a vision camera. In yet another aspect, the vision camera monitors the area being cut by the laser beam.

In still another aspect, the invention further comprises a beam splitter that reflects a portion of the reflected light to the polarizing beam splitter and transmits a portion of the reflected light to a sensor. One of the functions of this aspect is to monitor the cutting efficiency of a laser cutting beam to determine whether the cutting beam is cutting entirely through the wall of stent tube when the laser cutting beam is used to cut a stent pattern into the stent tube.

In an alternative aspect, the sensor provides a signal having a characteristic that is a function of an intensity of the reflected light, the characteristic being an indication of a degree of cut-through of the wall of the stent tube by the laser cutting beam. In still another alternative aspect, the characteristic is used by a process or in communication with the cutting laser to control the cutting laser.

In one alternative aspect, the invention includes a system of monitoring the cutting efficiency of a laser cutting beam to determine whether the cutting beam is cutting entirely through the wall of stent tube when the laser cutting beam is used to cut a stent pattern into the stent tube, comprising: a optical image lens to image the end view of the stent tube and to collecting the scattered laser light at the cutting regine; and a CCD camera to receive the image of the tube end view and scattered laser light.

In an alternative aspect, the CCD camera image provides a signal having a characteristic that is a function of an intensity of the scattered light, the characteristic being an indication of a degree of cut-through of the wall of the stent tube by the laser cutting beam. In still another alternative aspect, the characteristic is used by a process or in communication with the cutting laser to control the cutting laser.

In yet another aspect, the invention includes a method of monitoring a laser cutting process, comprising: focusing a laser cutting beam having selected characteristics upon a stent tube to cut a stent pattern into the stent tube; and receiving light reflected from the stent tube in a sensor, the sensor providing a signal representing certain characteristics of the cutting laser beam and/or optical components of the laser system.

In one alternative aspect, the sensor is a beam mapper. In yet another alternative aspect, the sensor is a laser beam profiler. In still another alternative aspect, the sensor is a vision camera. In yet another alternative aspect, depending on the type of sensor, the signal represents laser beam characteristics or a degree of cut-through of a wall of the stent tube.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a partial view of a stent showing various elements of a stent pattern.

FIG. 1A is a cross-sectional view of a portion of one of the elements of the stent pattern.

FIG. 2 is a side view of a typical arrangement of a computer controlled cutting station for cutting stent patterns into suitable tubing using a laser beam.

FIG. 3 is a schematic drawing showing various components of one embodiment of a system incorporating principles of the present invention, including a laserCam, beam profiler and DVT camera.

FIG. 4 is photograph showing an example of use of a LaserCAM in accordance with the embodiment of FIG. 3.

FIG. 5A is a series of images illustrating the use of one component of a system in accordance with FIG. 3.

FIG. 5B is a series of images illustrating the use of a laser camera of a system in accordance with FIG. 3.

FIG. 5C is a comparison of images taken by the laser camera of FIG. 5B comparing a beam profile produced by a clean optical chain and a beam profile produced by an optical chain having a defective ¼ wavelength plate.

FIG. 6 is a photograph showing an example of use of beam map system beam profiler as set forth in FIG. 3.

FIG. 7 shows an example of a beam map for a reflected laser beam generated using the system of FIG. 3.

FIG. 8 is a schematic drawing illustrating embodiment of the system of FIG. 3 including additional components such as a vision camera.

FIG. 9A is a partial side cut-away view showing a stent wall which has not been cut by a laser beam.

FIG. 9B is a partial side cut-away view showing a stent wall which has been partially cut through by a laser beam.

FIG. 9C is a partial side cut-away view showing a stent wall which has been completely cut through by a laser beam

FIG. 10 is a schematic drawing showing additional details of a vision system that may be used in accordance with the embodiments of FIGS. 1 and 8 to monitor the degree of cut through of the stent tube wall achieved by a laser beam impinging on the stent tube.

FIG. 11 depicts a series of photographs showing example images from the CCD camera in FIG. 10 when a stent wall which has not been cut by a laser beam, or has been partially cut through, or completely cut through by a laser beam.

### DETAILED DESCRIPTION OF THE PREFERRED EMBODIMENTS

FIG. 1 is an enlarged perspective view of a stent 10 illustrating an exemplary stent pattern and showing the placement of interconnecting elements 15 between adjacent radially expandable cylindrical elements. Each pair of the interconnecting elements 15 on one side of a cylindrical element is preferably placed to achieve maximum flexibility for a stent. In the embodiment shown in FIG. 1, the stent 10 has three interconnecting elements 15 between adjacent radially expandable cylindrical elements which are 120 degrees apart. Each pair of interconnecting elements 15 on one side of a cylindrical element are offset radially 60 degrees from the pair on the other side of the cylindrical element. The alternation of the interconnecting elements results in a stent which is longitudinally flexible in essentially all directions. Various configurations for the placement of interconnecting elements are possible. However, as previously mentioned, all of the interconnecting elements of an individual stent should be secured to either the peaks or valleys of the undulating structural elements in order to prevent shortening of the stent during the expansion thereof.

The number of undulations may also be varied to accommodate placement of interconnecting elements 15, for example, at the peaks of the undulations or along the sides of the undulations as shown in FIG. 1.

As best observed in FIG. 1, cylindrical elements in this exemplary embodiment are shown in the form of a serpentine pattern. As previously mentioned, each cylindrical element is connected by interconnecting elements 15. The serpentine pattern is made up of a plurality of U-shaped members 20, W-shaped members 25, and Y-shaped members 30, each having a different radius so that expansion forces are more evenly distributed over the various members.

The afore-described illustrative stent 10 and similar stent structures can be made in many ways. However, the preferred method of making the stent is to cut a thin-walled tubular member, such as, for example, stainless steel tubing to remove portions of the tubing in the desired pattern for the stent, leaving relatively untouched the portions of the metallic tubing which are to form the stent. In accordance with the invention, it is preferred to cut the tubing in the desired pattern by means of a machine-controlled laser, as exemplified schematically in FIG. 2.

The tubing may be made of suitable biocompatible material such as, for example, stainless steel. The stainless steel tube may be Alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants. Other biomaterials may also be used, such as various biocompatible polymers, co-polymers or suitable metals, alloys or composites that are capable of being cut by a laser.

Another example of materials that can be used for forming stents is disclosed within U.S. application Ser. No. 12/070,646, the subject matter of which is intended to be incorporated herein in its entirety, which application discloses a high strength, low modulus metal alloy comprising the following elements: (a) between about 0.1 and 70 weight percent Niobium, (b) between about 0.1 and 30 weight percent in total of at least one element selected from the group consisting of Tungsten, Zirconium and Molybdenum, (c) up to 5 weight percent in total of at least one element selected from the group consisting of Hafnium, Rhenium and Lanthanides, in particular Cerium, (d) and a balance of Tantalum.

The alloy provides for a uniform beta structure, which is uniform and corrosion resistant, and has the ability for conversion oxidation or nitridization surface hardening of a medical implant or device formed from the alloy. The tungsten content of such an alloy is preferably between 0.1 and 15 weight percent, the zirconium content is preferably between 0.1 and 10 weight percent, The molybdenum content is preferably between 0.1 and 20 weight percent and the niobium content is preferably between 5 and 25 weight percent.

The stent diameter is very small, so the tubing from which it is made must necessarily also have a small diameter. Typically the stent has an outer diameter on the order of about 0.06 inch in the unexpanded condition, the same outer diameter of the tubing from which it is made, and can be expanded to an outer diameter of 0.1 inch or more. The wall thickness of the tubing is about 0.010 inch or less.

Referring now to FIG. 2, the tubing 50 is put in a rotatable collet fixture 55 of a machine-controlled apparatus 60 for positioning the tubing 50 relative to a laser 65. According to machine-encoded instructions, the tubing 50 is rotated and moved longitudinally relative to the laser 65 which is also machine-controlled. The laser selectively removes the material from the tubing and a pattern is cut into the tube. The tube is therefore cut into the discrete pattern of the finished stent.

The process of cutting a pattern for the stent into the tubing is automated except for loading and unloading the length of tubing. Referring again to FIG. 2, it may be done, for example, using a CNC-opposing collet fixture 55 for axial rotation of the length of tubing, in conjunction with a CNC X/Y table 70 to move the length of tubing axially relatively to a machine-controlled laser as described. Alternatively, the collet fixture may hold the tube at only one end, leaving the opposite end unsupported. The entire space between collets can be patterned using the laser. The program for control of the apparatus is dependent on the particular configuration used and the pattern to be cut by the laser.

Referring now to FIG. 3, there is shown a laser system 100 for cutting stent patterns that includes optical elements and devices for providing for beam alignment, process monitoring and process control. The optical design of the system is unique in that it allows for reflection of a laser beam along the same path as an entering beam and subsequent diversion of the reflected beam toward beam diagnostic systems that monitor beam properties, laser beam location, system cleanliness, and any optical defects that develop in the system.

A laser 105 generates a linearly polarized laser beam 110 that is directed through an optical chain toward a stent tube 160. The optical chain includes components such as an iris 115, various wave plates, lenses, mirrors, and nozzles. A half wavelength plate 120 alters the orientation of the incoming linearly polarized beam. The polarized beam in the input beam plane then passes through the polarization beam splitting cube (PBS) 125, and the beam that is polarized perpendicular to the input plane is reflected to a beam dump 130. As shown, the transmitted beam 112 is projected towards the stent tubing, and may pass through an additional iris 135. This beam may be reflected by mirror 140 to provide access to the stent tube 160 in which a pattern is to be cut.

The polarization of the laser beam is then translated into circular polarization as the beam passes through a quarter wavelength plate 145. Depending on system design requirements, the beam may then be directed through a focusing lens 150 to impinge onto the stent tube 160. In some systems, the laser optical system may be separated from stent tube 160 by a window or other transparent barrier 155 to prevent debris from the contaminating the optical system. Some systems may also include a nozzle (not shown) used to introduce a shielding fluid or gas through which the laser beam is directed as it travels to the surface of the stent tube 160 to cut the stent pattern into the tube.

A portion of the laser beam impinging upon stent tube 160 will be reflected back from the stent tube. Pathway 170 illustrates this path the reflected light takes as it travels towards minor 140. The reflected beam passes through the focusing lens 150 and the quarter wave length plate 145 and has a linear polarization that is approximately 90 degrees to the incoming linearly polarized beam. In this way, the incoming and the reflected beam will be perpendicularly polarized, allowing the reflected beam to travel the same path, as indicated by path 170, as the incoming beam, but in a reverse direction, to reach the polarization beam splitting cube (PBS) 125.

When the reflected beam enters PBS 125, it is reflected without transmission because of its polarization. Thus, the reflected beam 114 is diverted along a new optical chain toward minor 175 and focusing lens 180. Depending on the design of the system, the reflected laser beam may pass directly to a beam mapping system 195, or a portion of the beam may be reflected by partial reflecting minor 185 to H-cam vision device 190.

In another embodiment, mirror 140 may be selected such that it transmits a portion of the beam 199 reflected from the stent tube through an achromatic lens 200 and filter 205 to a DVT camera 210. The system may also include a beam block 165 to capture any light from the incoming laser beam that is not reflected by minor 140.

The beam mapping system 195 provides real-time processing of the beam in order to analyze beam properties, such as focus spot size, ellipticity (circularity), beam mode, beam position, astigmatism and intensity profile, and the like. An example of the type of information provided by beam mapping system 195 is illustrated by graphic 201.

The H-Cam or laser cam beam profiler 190 processes the laser beam in order to determine variation of beam properties that indicate beam location, beam size, power changes due to nozzle cleanliness and any optical defects in the optical chain. For example, beam location may be determined by assessing whether the beam is at the top-dead-center location of the stent tube. When the beam is located at that position, the reflected beam will have a particular beam profile that can be monitored and measured by the vision system software used to analyze the output of the beam profiler. Likewise, when the nozzle is dirty or when there are defects in the optical components of the optical chain, the beam will diverge from an ideal profile, which can be detected by the vision system. An example of the type of information provided by the H-cam or laser beam profiler 195 is illustrated by graphic 203.

It will be understood that beam mapping system 195 and H-Cam 190 include one or more sensors that allow real time viewing and analysis of the reflected laser beam. These sensors produce output which may be digital or analog that may then be processed and analyzed using software executing programming commands on either a dedicated processor or a general purpose processor that is being operated under the control of software commands to carry out the specific analysis of the data generated by the sensors that is desired by the operators of the system. In some embodiments, this may provide for reports and images that are generated by the processor and displayed to the system operators using visual displays, paper reports, or both.

The data output by the sensors may also be stored in a memory for later analysis. Alternatively, the entire system may be operated in a network environment where individual systems are polled and controlled, and the operating parameters and sensor data are stored in memory as the cutting process occurs.

FIG. 4 depicts a prototype of one embodiment of a laser cam 190. In this embodiment, laser beam 114 is reflected by mirror 185, and reflected beam 117 first passes through a converging lens 250, and then through an attenuator wheel 255 before passing to the camera system 260. The attenuator wheel 255 may be used to further modify beam 117 as it is directed toward the camera system 260 in order to match the intensity of the laser beam to the sensitivity capability of the camera or sensor of the camera system.

FIGS. 5A-C depict a series of images taken from a prototype system in accordance with one embodiment of the present invention that are examples of the types of images that can be generated by the vision systems incorporated into the embodiment of the system shown in FIG. 3. For example, FIGS. 5A and 5B provides images taken by DVT camera 210 (FIG. 5A) and H-cam camera 190 to detect tubing alignment because when the tubing is perfectly aligned, the reflected beam will have a profile that is different from the beam profile when the tubing is misaligned. Compared with the images from the DVT optical camera 210 (FIG. 5A), the images from the H-cam camera system 190 show increased resolution and sensitivity, and thus any changes in alignment of the optical system are more easily detectable.

Likewise, cleanliness and integrity of the beam optics can be determined by comparing the beam profile to a known beam profile with clean optics. Similarly, the beam profile will be degraded where there is a defect in one or more of the optical properties, as is shown in one of the images in FIG. 5C. It will be understood that while such images may be presented to an operator for manual assessment, assessment may also be done using appropriate software, which may provide an operator alerts or other indications indicating that beam adjustment or other corrective action needs to be taken. In some embodiments, where the defect sensed is severe enough to compromise quality of the stent cutting operation, the cutting operation may be halted until corrective action may be taken.

FIG. 6 provides another embodiment of the present invention that includes the a beam mapping vision system 195. This embodiment is similar to the embodiment depicted in FIG. 4, but in this embodiment, laser beam 114 (FIG. 3) is transmitted by mirror 185. Transmitted beam 118 passes first through an attenuator wheel 255 and then through converging lens 250 before impinging on camera 260.

FIG. 7 shows an example of a beam map generated for a reflected laser beam using the various embodiments of beam mapping system 195. The image or images provided by the sensor or sensors comprising the beam mapping system are provided to a processor, which may be in operable communication with a memory that can be used for storing images, both as received from the sensor or sensors of the system, as well as information related to the images that may be the result of analysis of the image or images performed by the processor. The processor analyzes the image or images provided by the sensor using beam mapping software that provides for determining various beam properties for the laser beam from the images provided by the system sensor or sensors, such as ellipticity (circularity), power distribution, and other important beam characteristics, spot size, beam mode, beam position, astigmatism, and the like. The software may include instructions for producing various kinds of reports concerning the properties of the beam, and that report or reports may be displayed on a display visible by an operator, or may be provided to a printer for producing a hard report. The information associated with the report or reports may also be stored in the memory.

FIG. 8 illustrates another embodiment of the system as depicted in FIG. 3, with the addition of several other components to enhance performance of the system. For example, an imaging lens system 300 may be provided to view, in real time, the beam cutting through the thickness of the stent tube 160. This allows viewing of the position of the laser beam to ensure that it is impinging the tube at the tube's top dead center, as well as providing an image for use in monitoring and/or determining the thickness of the wall of the stent tube. The imaging lens system 300 shown includes a sensor or camera 302 that provides the image of the cutting action to a monitor. Alternatively, the image may be provided to a processor, which may be same processor described above, or a different processor, for analysis. The image may also be stored in a memory, or used to provide a hard copy report of the cutting activity. The sensor may also be capable of providing a video record of the cutting activity. Imaging lens system 300 may also include a collimation/focus tube assembly 304 and a lens 303 to allow for changing the viewing field and/or focus point of the sensor or camera 302. It will be appreciated that the invention is useful with or without these other components.

Those skilled in the art will appreciate that although the monitoring process may be performed in real-time during the cutting of the stent pattern into the stent tubing, it is also possible to stop the cutting process in order to monitor and adjust the laser optical system. For example, a beam monitoring optical chain may be inserted that includes a parabolic minor 301 in place of a stent tube, such as is shown in path 170. This mirror reflects the laser beam more efficiently than a stent tube typically will, and thus, the swap of these components can be used to provide for more accurate and precise monitoring and adjustment of the laser system optics.

FIGS. 9A-9C illustrate how the intensity of the reflection of the laser beam varies as a function of the condition of the surface of the stent tube 315. Depending on the condition of the surface of the stent tube in the area cut by laser beam 105, the laser beam will be reflected by the stent tube. The reflected beam returns along the path from which it arrived at the surface of the stent tube until it encounters mirror 140. Partially transmitted beam 199 travels through mirror 140 towards DVT camera 210 (FIG. 3).

As shown in FIG. 9A, if the stent tube has not been cut by the laser cutting beam 310, laser beam 105 will be strongly reflected backwards from the surface of the stent tube. If however, the stent tube 315 has only been partially cut through, as shown in FIG. 9B, the intensity of the reflected beam 199 will be weaker than if the surface was not cut through at all. The depth of the cut may be determined as a function of the intensity change from an expected reflected intensity of a non-cut tube surface. Where there is complete cut-through of the stent tube 315, as shown in FIG. 9C, substantially all of beam 105 will pass through the cut out area, and there will be minimal, if any, reflectance of laser beam. This change in intensity is analyzed by the processor from the images provided by DVT camera 210. Thus, the processor can provide a real time analysis of the not only the depth of cut, but also the time required for cutting a stent pattern out of the stent tube, and can provide information relating to cutting of the stent to an operator of the system, either through a real time display, alerts or other messages, and the like.

FIG. 10 is a schematic drawing showing additional details of a vision system 300 that may be used in accordance with the embodiments of FIGS. 1 and 8 to monitor the degree of cut through of the stent tube wall achieved by a laser beam impinging on the stent tube. In this system, a laser cutting beam 305 is focused by focusing lens 310 onto the surface of a stent tube 315 to cut a stent pattern into the sten tube. As described previously, the stent tube is rotated while the stent pattern is being cut into the stent tube. Thus, portions of the stent pattern that have been cut by the laser cutting beam are moved out of the path of the laser cutting beam so as to expose uncut areas of the stent tube to the laser cutting beam.

An imaging lens 320 images the end view of the stent tube 315 on to a CCD camera 325 during laser cutting process. This imaging system also collects scattered laser light from the laser cutting regime.

FIG. 11 illustrates exemplary images from CCD camera 325 when a stent wall has not been cut by a laser beam, or has been partially cut through, or completely cut through by a laser beam. When the laser beam does not cut through the stent tube wall, only small amounts of laser light is scattered until the laser beam cuts through the tube wall, wherein large amount of laser light is scattered into the inside of the tube.

Monitoring the cut-through using such a system and method is advantageous particularly where a pico-second or other short pulse laser is used to cut the stent pattern into the stent tube. In previous systems, using long pulse laser cutting, the waste pieces (islands) left behind as the pattern was cut into the stent would fall out of the tube, and it was obvious that the pattern was fully cut. However, due to the tightly focused beam of a short pulse or pico-second laser, the islands are typically still in place on the tube after cutting, which has been found to improve the dimensional accuracy of the cut. However, this precludes a guarantee that the pattern has been cut entirely through the wall of the stent tube, and is not discovered until post-proces sing of the stent begins. Utilizing the system and method of the various embodiments of the system illustrated in FIG. 10 allows for monitoring of cut-through in real time, and allows an operator to reject the stent at the cutting station. Moreover, a processor may monitor the laser operating parameters and adjust those parameters to ensure total cut-through in the event that the processor determines that the cutting efficiency of the cutting laser has decreased.

Other modifications and improvements may be made without departing from the scope of the invention. Accordingly, it is not intended that the invention be limited, except as by the appended claims.

We Claim:

1. A system for monitoring the cutting of a stent pattern into a stent tube using a laser beam, comprising:
a laser for generating a laser beam;
a polarizing beam splitter;
a quarter wave plate for imparting circular polarization to the laser beam;
a focusing lens for focusing the circularly polarized laser beam onto a tent tube into which a stent pattern is to be cut; and
a laser beam analyzer for analyzing a laser beam reflected by the stent tube back along the path of the laser beam and into the polarizing beam splitter, where the reflected beam is deflected into the laser beam analyzer.

2. The system of claim 1, wherein the laser beam analyzer is a beam mapper.

3. The system of claim 1, wherein the laser beam analyzer is a laser cam.

4. The system of claim 1, wherein the laser beam analyzer is a vision camera.

5. The system of claim 4, wherein the vision camera monitors the area being cut by the laser beam.

6. The system of claim 1, further comprising a beam splitter that reflects a portion of the reflected light to the polarizing beam splitter and Transmits a portion of the reflected light to a DVT camera.

7. The system of claim 6, wherein a DVT camera provides a signal having a characteristic that is a function of an intensity of the reflected light, the characteristic being an indication of a degree of cut-through of the wall of the stent tube by the laser cutting beam.

8. A system of monitoring the cutting efficiency of a laser cutting beam to determine whether the cutting beam is cutting entirely through the wall of stent tube when the laser cutting beam is used to cut a stent pattern into the stent tube, comprising:
a optical imaging lens; and
a CCD camera from receiving a scattered laser beam from the beam splitting minor, the reflected laser beam formed by reflection of the monitoring laser beam from the area of the stent tube cut by the cutting laser beam.

9. The system of claim 8, wherein CCD camera provides a signal having a characteristic that is a function of an intensity of the scattered light, the characteristic being an indication of a degree of cut-through of the wall of the stent tube by the laser cutting beam.

10. The system of claim 9, wherein the characteristic is used by a process or in communication with the cutting laser to control the cutting laser.

11. A method of monitoring a laser cutting process, comprising:
focusing a laser cutting beam having selected characteristics upon a stent tube to cut a stent pattern into the stent tube; and
receiving light reflected from the stent tube in a sensor, the sensor providing a signal representing certain characteristics of the cutting laser beam and/or optical components of the laser system.

12. The method of claim 11, wherein the sensor is a beam mapper.

13. The method of claim 11, wherein the sensor is a laser beam profiler.

14. The method of claim 11, wherein the sensor is a vision camera.

15. The method of claim 11 wherein the signal represents an degree of cut-through of a wall of the stent tube.
